(19) United States
(12) Patent Application Publication
(10) Pub. No.: US 2019/0142329 A1
(43) Pub. Date: May 16, 2019

(54) METHODS AND SYSTEM FOR DETECTING LIVING, NON-DEMENTED PATIENTS WHO HAVE ALZHEIMER'S DISEASE NEUROPATHOLOGY FOR ALZHEIMER'S DISEASE

(71) Applicant: INSPIRED LIFE MEDICAL INC., LeSueur, MN (US)

(72) Inventor: Gregory B. Mills, Kansas City, KS (US)

(21) Appl. No.: 16/189,870

(22) Filed: Nov. 13, 2018

Related U.S. Application Data
(60) Provisional application No. 62/585,628, filed on Nov. 14, 2017.
(60) Provisional application No. 62/749,465, filed on Oct. 23, 2018.

(51) Int. Cl. A61B 5/00

(52) U.S. Cl. CPC A61B 5/4088; C07K 14/4711; A61B 5/4011

(57) ABSTRACT

The present method is directed to determining whether a living, non-demented patient with Alzheimer's neuropathology, i.e., significant levels of amyloid plaque in the brain, is within the early pre-clinical stages of Alzheimer's disease. In turn, if the living, non-demented patient with Alzheimer's neuropathology is determined to not be in the early stages of Alzheimer's disease, then it may be concluded that the patient has at least a resistance to Alzheimer's disease neuropathology, and perhaps an immunity thereto, wherein the amyloid plaque does not localize to the patient's brain synapses, thereby preserving cognitive ability. A 3-step method comprising (1) conducting a cognitive test; (2) detecting brain plaque levels; and (3) conducting a bi-lateral olfactory sensitivity test using pure odorants is therefore provided.

### CROSS-REFERENCE TO RELATED APPLICATIONS

This application claims the benefit of U.S. Provisional Application Ser. No. 62/585,628, filed Nov. 14, 2017 and entitled METHODS OF DIAGNOSING ALZHEIMER'S DISEASE AND AROMA PRESENTATION DEVICES and U.S. Provisional Application Ser. No. 62/749,465, filed Oct. 23, 2018 and entitled METHOD OF DIAGNOSING LIVING NON-DEMENTED INDIVIDUALS WITH ALZHEIMER'S NEUROPATHOLOGY, the entire contents of each of which are hereby incorporated by reference.

### FIELD OF THE INVENTION

The present invention generally relates to devices, systems and methods for determining whether a living, non-demented patient who has neuropathology commonly associated with Alzheimer's disease may actually have Alzheimer's disease.

### DESCRIPTION OF THE RELATED ART

It is known that Alzheimer's disease may begin before clinical symptoms with deterioration of the left olfactory bulb. It is possible, therefore, to evaluate a patient's olfactory sensitivity, specifically to identify any significant differential between the sensitivities of the patient's left olfactory bulb and the right olfactory bulb. Thus, applicant has discovered that it is possible to use “pure odorants” defined herein as those substances that stimulate only cranial nerve 1 and do not stimulate cranial nerve 5 to very specifically test the differential, if any exists, between the left and right olfactory bulbs. See, e.g., U.S. Pat. No. 9,717,454, assigned to applicant and entitled Method of Ruling Out Alzheimer's disease, the entirety of which in hereby incorporated by reference. There, the method for administering one or more pure odorants in succession to each nostril is described. The left nostril corresponds with the left olfactory bulb and the right nostril corresponds with the right olfactory bulb. If the patient's olfactory sensitivity is lower for the left olfactory bulb (left nostril) than the right olfactory bulb (right nostril) for the administered pure odorants, then it is concluded that the left olfactory bulb is more damaged than the right olfactory bulb and that it is likely that the patient is in very early stages of Alzheimer's disease. On the other hand, if there is no difference in olfactory sensitivity between the left and right olfactory bulbs for the administered pure odorants, then it is concluded that there is no difference in damage between the left and the right olfactory bulbs and therefore Alzheimer's disease may be excluded as a diagnosis.

Generally, Alzheimer's disease is associated with amyloid plaque and tau as a diagnostic, a suspected mechanism of injury (loss of cognitive abilities) and a target for treating Alzheimer's disease. Thus, passing a cognitive screen is generally thought to be sufficient to rule out Alzheimer's disease and, therefore, these patients are not typically tested for the presence of amyloid plaque. These patients may, therefore, be at risk for developing Alzheimer's disease if, e.g., as in high cognitive functioning people, the cognitive decline associated with Alzheimer's disease is delayed, sometimes for quite a long period of time followed by a rapid loss of cognition. It would be advantageous to confirm whether these patients are in fact in the very early and pre-clinical stages of Alzheimer's disease.

Moreover, recently some non-demented, non-MCI people have been tested for the presence of amyloid plaque and found to have a significant load of plaque—yet they don't show any cognitive decline. These patients may also be in the early pre-clinical stages of Alzheimer's disease and, therefore, it would be advantageous to confirm if this is the case or not.

One research study has evaluated brains of individuals with no MCI but who also have significant amyloid plaque loads, characterizing these patients as Non-Demented with Alzheimer' Neuropathology (“NDAN”). The authors began by noting that the NDAN patients did not have significant levels of amyloid plaque in the region of synapses, unlike the patients with Alzheimer's disease. They next analyzed the NDAN brains by testing slices taken from the hippocampus for the presence or absence of certain proteins, a process repeated for patients who did have Alzheimer's disease and for controls that were ruled out for Alzheimer's disease by virtue of not being demented and not having significant amyloid plaque loads. The study concluded by determining that a total of fifteen proteins were expressed in a uniquely different pattern in the NDAN patients as compared with both the Alzheimer's disease patients and the control and hypothesized that perhaps these fifteen proteins may provide the basis for resisting localization of amyloid plaque within synapses, thus allowing NDAN patients to retain normal cognition levels despite the amyloid plaque load. See Zolochevska, O., et al., Postsynaptic Proteome of Non-Demented Individuals with Alzheimer's Disease Neuropathology, Journal of Alzheimer's Disease 65 (2018) 659-682.

It would be highly advantageous to provide a method that confirms whether NDAN patients are, or are not, actually in the early stages of Alzheimer's disease.

The present invention addresses these, among other, needs.

### BRIEF SUMMARY OF THE INVENTION

The present method is directed to determining whether a living, non-demented patient with Alzheimer's neuropathology, i.e., significant levels of amyloid plaque in the brain, is within the early pre-clinical stages of Alzheimer's disease. In turn, if the living, non-demented patient with Alzheimer's neuropathology is determined to not be in the early stages of Alzheimer's disease, then it may be reasonably concluded that the patient has at least a resistance to Alzheimer's disease neuropathology, and perhaps an immunity thereto, wherein the amyloid plaque has not localized to the patient's brain synapses, thereby preserving cognitive ability. A 3-step method comprising (1) conducting a cognitive test; (2) detecting brain plaque levels; and (3) conducting a bi-lateral olfactory sensitivity test using pure odorants is therefore provided.

### BRIEF DESCRIPTION OF THE DRAWINGS

FIG. 1 illustrates a flowchart of a testing method according to the present invention.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

The present system is directed in various embodiments to devices, systems and methods for detection, evaluation and/or monitoring olfactory dysfunction by measuring and determining the patient's olfactory detection threshold for the left and the right nostril. More specifically, the present invention relates to devices, systems and methods for detecting an asymmetric differential in a patient's relative olfactory detection threshold (left vs right nostril) which, when present, may be used as a device to detect, diagnose and/or monitor olfactory deterioration resulting from Alzheimer's disease. Alternatively, general olfactory deterioration may be tested and detected using the present invention.

### Definitions

As used herein, “symmetric” or “symmetrical” means that there is not a significant differential in the subject patient's ability to detect and/or identify odors between odors administered and/or inhaled into the patient's left nostril vs. the patient's right nostril as measured by the olfactory threshold determined for each nostril.

As used herein, “asymmetric” or “asymmetrical” means that there is a significant asymmetry or differential in the subject patient's ability to detect and/or identify odors between odors administered and/or inhaled into the patient's left nostril vs. the patient's right nostril as measured by the olfactory threshold determined for each nostril.

“Olfactory dysfunction” is defined herein as a patient with a disorder and/or condition with one or more of the following: hyperosmia, hyposmia, anosmia, and dysosmia. The olfactory dysfunction may be symmetric or asymmetric as those terms are defined herein.

“Pure odorant”, also referred to equivalently as “pure aroma” is defined as substances including molecules and/or compounds which principally stimulate the olfactory cell receptors associated with the olfactory nerve, aka the first cranial nerve, and that do not trigger or excite the trigeminal nerve associated with the fifth cranial nerve. A non-exhaustive and categorized listing of pure odorants follows:

The Pure Odorant Spice Family Comprises:

Cinnamon;

Clove;

Vanilla;

Nutmeg; and

Allspice.

The Pure Odorant Food Family Comprises:

Peanut/Peanut Butter;

Coffee;

Cocoa;

Apple;

Almond (bitter); and

Strawberry.

The Pure Odorant Herbal Family Comprises:

Peppermint;

Spearmint;

Wintergreen;

Allspice;

Parsley;

Sage;

Turmeric;

Thyme;

Basil;

Dill weed;

Caraway;

Anise;

Fennel;

Mace;

Palmarosa; and

Patchouli.

The Pure Odorant Floral Family Comprises:

Rose;

Lemongrass;

Rosemary;

Lavender;

Lilac;

Violet; and

Origanum.

The Pure Odorant Citrus Family Comprises:

Orange;

Tangerine;

Lemon;

Lime;

Mandarin;

Grapefruit;

Bergamot and

Petitgrain.

The Pure Odorant Wood and Resin Based Family Comprises:

Eucalyptus;

Juniper berry;

Pine;

Tea tree;

Spruce;

Ho wood;

Cypress;

Cedar;

Birch;

Fir;

Cajeput;

Camphor;

Cassia;

Citronella;

Clary;

Copaiba;

Elemi;

Hydacheim;

Litsea; and

Niaouli.

“Pure odorant detection threshold” is defined as the point at which the concentration of pure odorant molecules saturate the olfactory nerve to the extent that a cognitive reaction first takes place. At this point, the subject patient is able to express that he or she is smelling something, but not necessarily able yet to identify the aroma by name. The pure odorant detection threshold may be found to be asymmetrical, i.e., significantly different as between the nostrils, indicating olfactory dysfunction. Alternatively, the pure odorant detection threshold may be found to be symmetrical between the tested nostrils.

“Pure odorant identification threshold” represents a slightly longer latent period than the “pure odorant detection threshold” as it is defined as the point at which the patient is able to actually identify the pure odorant by name, indicating that cognitive processing has occurred.

“Odorant” is defined herein as a compound that does trigger the trigeminal nerve.

“Effective amount” of the odorant or pure odorant is defined as the amount of pure odorant required to infuse the aroma airway passage during operation of the various devices, systems and methods of the present invention sufficiently to enable a patient to smell the pure odorant, i.e., when saturation of the olfactory nerve is sufficient to enable the reaching of the pure odorant detection threshold for the patient and nostril being tested.

“Clear air”, also referred to as pure or atmospheric air, is defined as air that does not comprise the odorant used in the inventive embodiments of the present invention. Preferably, clear air comprises air that is substantially uncontaminated by any odorant, including pure odorants. Clear air may comprise ambient air, i.e., atmospheric air, either filtered or unfiltered, or air that is provided from a clear air source such as an air tank or nebulizer and/or from a mechanized powered air pump as is well known in the art.

Aroma detection testing is done for many reasons and in a number of ways to accomplish various purposes, including diagnostic medical tests. Odor identification for example, comprise a common olfactory function that is tested. Numerous odor identification devices and methods have been developed to screen for what might be called a “scent memory function”. This sort of testing metric might be called “Tell me what you smell?” This corresponds to the presently defined “pure odorant identification threshold”. This endpoint for testing has several serious problems, not the least of which is the required inclusion of cognitive identification of the aroma or odor presented.

Pure aroma olfactory detection threshold response time is of particular clinical interest, especially when done bilaterally. In other words, the same aroma sensing test is preformed separately on each nostril and the test results compared, left nostril vs right nostril. This is done without aroma identification even being required. Perhaps the metric could be called, “Tell me when you smell something.”

The bilateral olfactory sensitivity screening testing with pure odorants may be conducted using a very simple test device, e.g., a container that is opened to allow the patient to smell the pure odorant therein. Alternatively, more precise measurements of the bilateral olfactory sensitivities of the right vs left olfactory bulb may be made using more precise and controlled devices such as those described in various patent applications to applicant.

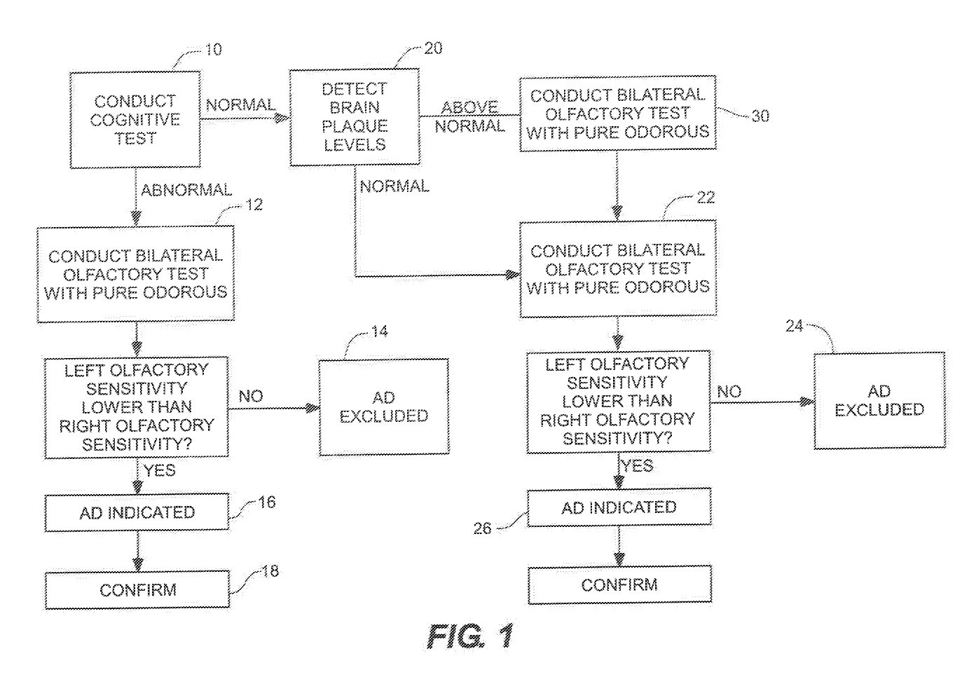

Referring now to FIG. 1, a methodological flow chart is provided to evaluate living persons for the likely presence or exclusion of Alzheimer's disease. The method initiates at step 10 with a cognitive test or evaluation as is well known in this art to determine if the patient's cognitive level or cognition is normal or abnormal, i.e., below the normal levels of cognition. If the patient's cognition is evaluated as abnormally low, then a bilateral olfactory sensitivity screening test is administered using pure odorants as discussed above at step 12. If the results of this test indicate no difference between the left and right nostrils (corresponding with the left and right olfactory bulbs, respectively), then the patient is excluded from a diagnosis of Alzheimer's disease 14. Similarly, if the results of the patient's right side testing show a lower sensitivity to the administered pure odorants than the patient's left nostril/olfactory bulb, then the patient is excluded from having Alzheimer's disease. However, if the patient's left nostril/left olfactory bulb result in lower sensitivity to the administered pure odorants than the patient's right nostril and right olfactory bulb, then the results indicate the patient may have early stage Alzheimer's disease 16 and further confirmation is indicated by, e.g., MRI 18.

If the cognition test results are not abnormal, then the patient moves to step 20 for plaque detection in the brain using known methods and techniques. If the plaque detection results are normal, then a bilateral olfactory sensitivity testing screen using pure odorants as described above is administered at 22 as discussed above. If the results of this test indicate no difference between the left and right nostrils (corresponding with the left and right olfactory bulbs, respectively), then the patient is excluded from a diagnosis of Alzheimer's disease 24. Similarly, if the results of the patient's right side testing show a lower sensitivity to the administered pure odorants than the patient's left nostril/olfactory bulb, then the patient is excluded from having Alzheimer's disease. However, if the patient's left nostril/left olfactory bulb result in lower sensitivity to the administered pure odorants than the patient's right nostril and right olfactory bulb, then the results indicate the patient may have early stage Alzheimer's disease 26 and further confirmation is indicated by, e.g., MRI 28.

The flow sequence from 20 through 28 are important even though the patient's testing (cognition and plaque levels) are normal to this point, in order to confirm that the patient is not in very early stages of Alzheimer's disease.

Further, if the patient's plaque levels are greater than normal, then the bilateral olfactory screening testing using pure odorants as discussed above is administered and further according to steps 20 through 28 to determine if the patient may have early stage Alzheimer's disease (left side olfactory bulb sensitivity lower than right side olfactory bulb sensitivity) or if the patient may be excluded from a diagnosis of Alzheimer's disease.

In each case where the cognition test is normal and plaque detected at a level that is greater than normal, and the patient's bilateral olfactory screening test using pure odorants indicates no difference (right vs left), i.e., is normal, then it is concluded that the amyloid plaque is not localizing within the patient's brain synapses based on normal cognition and olfactory sensitivity results in combination with Alzheimer's disease pathology. It may further be concluded that the patient comprises a resistance to Alzheimer's disease based on normal cognition and olfactory sensitivity results in combination with Alzheimer's disease pathology. Based on these test results and in combination with the Zolochevska et al., paper discussed above, it may be further concluded that the patient may have unique expression of the at least fifteen proteins in the NDAN patient and that may function to protect the patient from Alzheimer's disease by protecting the patients neuronal synaptic connections in the brain from the presence of amyloid plaque. Indeed, the presence of such protective proteins in NDAN patients with normal bilateral olfactory screening test results with pure odorants may indicate that these patients are perhaps immune to the onset of Alzheimer's disease.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

1. A method for confirming that a living, non-demented person with Alzheimer's neuropathology may be excluded from a diagnosis of Alzheimer's disease, comprising:
testing the patient's cognitive level and determining the patient's cognitive level is normal;
detecting the patient's brain plaque level and determining the patient's brain plaque level is above normal;
testing the patient's bilateral olfactory sensitivity by administering pure odorants to a first nostril of the patient, then administering the pure odorants to the second nostril of the patient to determine if the patient's left olfactory bulb is less sensitive to the pure odorants than the patient's right olfactory bulb;
concluding that, if the patient's left olfactory bulb is not less sensitive to the administered pure odorants, then the patient may be excluded from a diagnosis of Alzheimer's disease; and
concluding that, if the patient's left olfactory bulb is less sensitive to the administered pure odorants, then the patient is not excluded from a diagnosis of Alzheimer's disease.

2. A method for confirming that a living, non-demented person with Alzheimer's neuropathology does not comprise localization of amyloid plaque within brain synapses, comprising:
testing the patient's cognitive level and determining the patient's cognitive level is normal;
detecting the patient's brain plaque level and determining the patient's brain plaque level is above normal;
testing the patient's bilateral olfactory sensitivity by administering pure odorants to a first nostril of the patient, then administering the pure odorants to the second nostril of the patient to determine if the patient's left olfactory bulb is less sensitive to the pure odorants than the patient's right olfactory bulb;
concluding that, if the patient's left olfactory bulb is not less sensitive to the administered pure odorants, then localizing of amyloid plaque is not present within the patient's brain synapses; and
concluding that, if the patient's left olfactory bulb is less sensitive to the administered pure odorants, then localizing of amyloid plaque is present within the patient's brain synapses.

3. A method for confirming that a living, non-demented person with Alzheimer's neuropathology is resistant to Alzheimer's disease, comprising:
testing the patient's cognitive level and determining the patient's cognitive level is normal;
detecting the patient's brain plaque level and determining the patient's brain plaque level is above normal;
testing the patient's bilateral olfactory sensitivity by administering pure odorants to a first nostril of the patient, then administering the pure odorants to the second nostril of the patient to determine if the patient's left olfactory bulb is less sensitive to the pure odorants than the patient's right olfactory bulb;
concluding that, if the patient's left olfactory bulb is not less sensitive to the administered pure odorants, then the patient is resistant to Alzheimer's disease; and
concluding that, if the patient's left olfactory bulb is less sensitive to the administered pure odorants, then the patient is not resistant to Alzheimer's disease.

4. A method for detecting a living, non-demented person with Alzheimer's neuropathology (“NDAN”), comprising:
testing the patient's cognitive level and determining the patient's cognitive level is normal;
detecting the patient's brain plaque level and determining the patient's brain plaque level is above normal;
testing the patient's bilateral olfactory sensitivity by administering pure odorants to a first nostril of the patient, then administering the pure odorants to the second nostril of the patient to determine if the patient's left olfactory bulb is less sensitive to the pure odorants than the patient's right olfactory bulb;
concluding that, if the patient's left olfactory bulb is not less sensitive to the administered pure odorants, then the patient is detected as NDAN.
